(19) United States
(12) Patent Application Publication
(10) Pub. No.: US 2011/0319607 A1
(43) Pub. Date: Dec. 29, 2011

(54) Method For The Microbial Production Of A Body Containing Cellulose

(75) Inventors: Gunter Bertholdt, Heiningen (DE); Dirk Weuster-Botz, Dachau (DE); Michael Hofinger, Burgkirchen (DE); Katharina Seiffe, Munchen (DE)

(73) Assignee: BIOREGENERATION GMBH, Neuried (DE)

(21) Appl. No.: 13/127,066

(22) PCT Filed: Nov. 9, 2009
(86) PCT No.: PCT/EP2009/007998

§ 371 Date: Jun. 21, 2011

(30) Foreign Application Priority Data
Nov. 7, 2008 (DE) 102008056413.3

(51) Int. Cl. C08B 37/00; C12M 1/12; C12P 19/04

(52) U.S. Cl. 536/30; 435/101; 435/297.1

(57) ABSTRACT

The invention relates to a method for producing a body (7) comprising cellulose with the help of cellulose-producing organisms, which are supplied with a nutrient solution. A particularly uniform structure and density of the cellulose bodies can be achieved when a vessel (1) having a membrane (4) is supplied, said membrane being permeable to the nutrient solution and is substantially impermeable to the organisms, wherein the organisms are cultivated on one side of the membrane (4), where a gas atmosphere prevails, and the nutrient solution is arranged on the other side of the membrane and passes through the membrane (4) in the direction of the organisms, to supply them.

### BACKGROUND OF THE INVENTION

The invention relates to a method for producing a body comprising cellulose, as well as a body made of cellulose, which is produced according to this method. Finally, the invention relates to an arrangement for producing a body made of cellulose.

### STATE OF THE ART

It is known from the state of the art that microorganisms can form cellulose. The bacterium Gluconacetobacter xylinus (in the past called Acetobacter xylinum), for example, synthesizes pure cellulose in a batch culture at the boundary layer between a nutrient medium and air, which is yielded extracellularly. These very long-chain cellulose molecules congregate into bands, so-called “ribbons”, wherein due to hydrogen bonds and hydration highly crystalline structures are formed. Therefore, this cellulose is also called microcrystalline cellulose. The bands become felted and macroscopically form a solid layer with a gel-to cartilage-like consistency.

It could be demonstrated that this microcrystalline cellulose of bacterial origin is not degraded in mammalian tissue and also does not induce a foreign body reaction. Hollow bodies made of micro-crystalline cellulose have already been implanted as vascular grafts into the carotid artery of a rat with satisfactory results. In that, micro-crystalline cellulose proved to be an inherently stable biocompatible material.

It is known from the state of the art to cultivate cellulose-forming bacteria in a batch culture at the boundary layer between an aqueous nutrient medium and air. In that, a cellulose layer floating on the nutrient medium is formed (the so-called “pellicle”). In the German part DE 603 02 346 T2 of a European patent application it is suggested to use this pellicle as a wound dressing. In the production of cellulose bodies according to this method it showed that the reaction conditions in the batch culture are changing, because nutrients in the nutrient medium are used up and metabolites of the cellulose-forming bacteria accumulate in the nutrient medium. Thus, the pH of the nutrient medium can change. Furthermore, the altered reaction conditions may affect the quality of the formed cellulose, such that cellulose layers with an altered, inhomogeneous structure and density may be formed. In particular, an increasing solidification towards the air-facing side of the cellulose layer can be observed. The structural changes can be that pronounced that the formation of individual layers (delamination), which separate from one another with the impact of shearing forces, can be observed.

### THE OBJECT UNDERLYING THE INVENTION

Therefore, the invention is based on the object of creating a method using which cellulose bodies can be produced, which have a substantially more homogeneous structure and density. Furthermore, the invention is based on the object of providing an improved body made of cellulose. Finally, the invention is based on the object of creating an improved arrangement for producing such cellulose bodies.

### SOLUTION ACCORDING TO THE INVENTION

In order to solve the object, the invention teaches a method for producing a body containing cellulose, in which cellulose-forming organisms are arranged on one side of a membrane, where an oxygen-containing atmosphere prevails, and there form cellulose. The organisms are supplied with a nutrient solution, which is substantially provided on the other side of the membrane. In that, the membrane is substantially impermeable for the organisms and the cellulose, for the nutrient solution, however, it is permeable. Due to this characteristic of the membrane, the nutrient solution can pass through the membrane to the side of the organisms and there supply the organisms.

Furthermore, the invention teaches an arrangement for producing one or several cellulose bodies, comprising a vessel with a membrane, which is substantially impermeable for the organisms and the cellulose, however, permeable for the nutrient solution.

Using the method and apparatus according to the invention, cellulose bodies can be produced, which have a particularly homogeneous structure and density.

During the production process, the nutrient medium and/or the gas atmosphere are preferably exchanged or renewed, respectively, in order to provide uniform growth or reaction conditions, respectively, for the organisms, if possible. In that, in particular the concentration of nutrients and the pH at the site of the cellulose-forming organisms are of particular relevance.

The exchange of nutrient medium and/or gas atmosphere may be controlled. In this case, a control with a sensor system, like e.g. a pH sensor, and an actuator, like e.g. a controllable valve, which controls the supply or discharge of a medium, has to be provided. Beside the pH, other physical quantities may of course be controlled.

The nutrient solution preferably does not contain any cellulose-forming organisms. Due to the semi-permeable properties of the membrane, also during the production process, no cellulose-forming organisms get into the nutrient solution. Thus, in particular pellet formation in the nutrient solution and the formation of cellulose deposits in supply or discharge lines can be prevented.

The invention may, e.g., be used to produce cellulose-comprising medical products with a specified three-dimensional shape. In particular, the invention can be used to produce wound dressings as well as structured implants, e.g. vascular prostheses. The invention may also be used to produce cellulose bodies to be used for applications other than medical ones, e.g. for speaker membranes or electronic paper. For any applications stated, in particular the high homogeneity of the cellulose body achievable with the method according to the invention can be advantageous.

A preferred membrane consists of a hydrophilic material, preferably a polymer or an inorganic, non-metal material, which has passed through a thermal process, preferably ceramics. Preferred are membrane materials having pores, e.g. micro- or ultra-filtration membranes. With this embodiment of the invention it can be achieved that the pores fill with the nutrient medium by contact with the reservoir. With this embodiment of the invention it can be achieved that the capillary forces are sufficient to trans-port nutrient medium through the membrane. The membrane is preferably sterilizable with steam.

In a particular embodiment of the invention, only parts of the membrane are designed permeable for the nutrient solution. Therefore, one or several areas of the membrane can be determined, where one or several cellulose bodies are formed.

The membrane can be equipped with a support layer, e.g. a mesh, in order to support the membrane against the load of the cellulose body or bodies.

According to a first embodiment of the invention, the membrane is formed as a flat, substantially horizontal surface. In this case, the nutrient solution is substantially located underneath the membrane in a vessel, and the cellulose-forming organisms above the membrane. Due to the semi-permeable property of the membrane, the nutrient medium can penetrate upwards through the membrane and supply the organisms. The organisms, however, cannot pass into the nutrient solution. The membrane is preferably slightly immersed in the nutrient solution, such that on the membrane, a thin layer, e.g. with a thickness of a few mm, of nutrient solution is formed. Basically, it is sufficient already, if the nutrient solution wets the surface of the membrane.

According to a second embodiment of the invention, the membrane forms a peripheral surface of a hollow mold. This hollow mold may be, e.g., an elongated body, like e.g. a hollow cylinder, preferably a circular cylinder. In this embodiment, the cellulose-forming organisms are preferably located in the interior of the hollow mold, while the nutrient solution is provided outside the hollow mold. The reverse arrangement, however, is likewise possible. Again, the nutrient solution can diffuse through the membrane and supply the organisms located in the interior with nutrients. The interior of the hollow mold preferably is only partially filled with a suspension of cellulose-forming organisms. In another part of the interior, a gas atmosphere prevails.

The hollow mold may have an internal mold core, like e.g. a centrally arranged rod. The mold core may be irreversibly deformable, like described, e.g., in the German Patent Applications DE 10 2007 006 843 and DE 10 2007 006 844. The entire contents of the above documents in this respect shall be part of the present disclosure by reference. The hollow cellulose body may at least partially be produced according to the method disclosed in the German Patent Application DE 10 2007 016 852. The entire contents of the above document in this respect shall be part of the present disclosure by reference. The membrane may also be integrated into the wall of a hollow mold, e.g. by forming the base, on which the mold is established in layers according to the method of DE 10 2007 016 852. Several interior spaces can be provided, too. This may advantageously facilitate the production of larger item numbers. Preferably, all interior spaces are substantially formed equally.

In the interior of the hollow mold, preferably there also is a gas, in particular an oxygen-containing gas, like e.g. air, in order to supply the organisms with the gas required for the growth process.

According to a preferred embodiment of the invention, the passage of the nutrient medium through the membrane is controlled. This may be achieved, e.g., via variation of the gas pressure, in particular the partial pressure of the water vapor in the interior of the hollow mold and/or the hydrostatic pressure of the nutrient solution. Therewith, a substantially uniform transport rate of nutrients to the cellulose-forming organisms can be achieved. The hydrostatic pressure at the membrane may, e.g., be adjusted by immersing the hollow mold at different depths into the reservoir of the nutrient medium.

According to a particular embodiment of the invention, the elongated hollow mold is immersed into the nutrient liquid that far that substantially the entire outside of the membrane is in contact with the nutrient solution. Preferably, the entire hollow mold is immersed into the nutrient liquid reservoir. In the interior of the hollow mould, preferably an excess gas pressure prevails, in order to achieve that it does not fill up with nutrient liquid, but that only such amount of nutrient liquid penetrates through the membrane, as is desired for the supply of the cellulose bacteria.

According to another embodiment of the invention, the elongated hollow mold is immersed into the nutrient liquid reservoir that far that only part of the membrane is in contact with nutrient liquid at any time. In the interior of the hollow mold, preferably normal pressure prevails.

The hollow mold preferably extends in horizontal direction. The direction of growth of the cellulose thus substantially runs transverse to the longitudinal direction of the hollow mold.

The hollow mold is preferably arranged rotatable around its longitudinal axis, in order to compensate for hydrostatic differences in pressure and the unequal influence of gravity on the cellulose growth on a time average. In this case, the hollow mold is driven in a slowly rotating manner during the production process.

In a preferred embodiment of the invention, a further membrane is provided in the interior of the hollow mold, which is substantially impermeable for the organisms and the cellulose, for a gas, however, in particular oxygen, it is permeable. Preferred materials for the second membrane are PTFE, FEP, PFA, TFA, PFP, TTE, TPE-E, MVQ, MFQ, PEEK, PET, TBT, PVC, PMMA, PPS, cellulose ester, polyamide, rubber, in particular Latex or natural rubber, polypropylene, polycarbonate, polystyrene, polyurethane or silicon. The second, inner membrane defines a hollow space together with the first, outer membrane, in which the cellulose is growing. In that, the contour of the membrane determines the later shape of the cellulose bodies produced. If the second membrane is arranged concentrically to the first membrane, for example, a circular hollow space is defined.

In this manner, e.g., blood vessel prostheses can be produced from cellulose. The hollow space is preferably enclosed in order to prevent penetration of contaminations. This is in particular advantageous for the production of cellulose bodies for medical purposes.

The second membrane is preferably sterilizable with steam. It may be a component of a solid body in order to thus achieve higher stability.

Such a second membrane or a membrane body, respectively, may in principle be used in any apparatus according to the invention, in order to provide the cellulose bodies with a desired shape. Thus, e.g., on the horizontally arranged membrane, a second membrane may be arranged, too, which together with the first membrane forms one or several hollow spaces, in which the cellulose bodies grow.

The two membranes are preferably glued together, welded together or otherwise connected in at least one point. The hollow space formed between the membranes may have several subdivisions. Preferably, each interior space is inoculated with cellulose-forming organisms. With this embodiment of the invention, a high number of cellulose bodies can be produced simultaneously.

The production of the cellulose bodies preferably takes place in a vessel, preferably a trough-like vessel. The vessel is preferably closed—apart from optional openings for the exchange of nutrient medium and/or gas. It preferably comprises a base part and a cover.

Once the cellulose body has reached the desired height or completely fills the hollow mold, respectively, it may be detached from the membrane. Subsequently, the cellulose bodies produced are cleaned in a known manner, e.g. as disclosed in DE 10 2006 007 412, the entire contents of which in this respect shall be part of the disclosure by reference. The cellulose preferably is microbial cellulose, particularly preferred microcrystalline cellulose. The cellulose-forming organisms preferably are bacteria, particularly preferred bacteria of the strain Gluconacetobacter xylinus. It is perceivable that other cellulose-forming microorganisms are used, too, e.g. suitable strains of Agrobacterium, Rhizobium, Sarcina, Pseudomonas, Achromobacter, Aerobacter and Cooglea. Since the genes of the cellulose-synthesizing enzyme complexes of Gluconacetobacter xylinus are known, these may also be introduced into other microorganisms, like e.g. Escherichia coli, applying known molecular-biological methods, whereby these organisms, too, may be considered as cellulose-forming organisms.

For cultivation of cellulose-forming bacteria, different nutrient media have been described. A suitable, frequently used medium is the medium of Schramm and Hestrin described in the Biochemical Journal 58 of 1954, pages 345-352. The entire contents of the above article in this respect shall be part of the present disclosure by reference. One disadvantage of this medium may consist in the fact that it is not exactly defined, since it contains yeast extract and peptone.

For the embodiment of the present invention, a fully synthetic medium is preferred, like e.g. described by Formg et al. in Applied and Environmental Microbiology of 1989, Volume 55, No. 5, pages 1317-1319. The entire contents of the above article in this respect shall be part of the present disclosure by reference. One disadvantage of this medium may consist in the slightly slower growth of the bacteria.

It is also perceivable to use the so-called kombucha mushroom for the invention. Beside Gluconacetobacter xylinus, this culture contains numerous other organisms living in symbiosis, like yeasts and bacteria, and can be maintained by a medium solely consisting of black tea and saccharose (100 g/l).

### BRIEF DESCRIPTION OF THE DRAWINGS

In the following, the invention is explained in more detail on the basis of schematic drawings and embodiments, wherein:

FIG. 1 shows a schematic view of the working principle of a first embodiment of the invention;

FIG. 2 shows a perspective view of an embodiment of the invention according to the principle shown in FIG. 1;

FIG. 3 is a representation of one of the two identical chambers of the embodiment according to FIG. 2, (a) in a perspective view, (b) from the top, (c) in a first lateral view, where the supply and discharge connections run in parallel to the image plane, (d) in a lateral view rotated by 90° compared to that, and (e) in a cross-sectional view through the chamber at the level of the supply and discharge connections;

FIG. 4 shows an embodiment of the invention, where the membrane forms a tubular interior space, in a cross-sectional view vertical to the longitudinal axis of the tube;

FIG. 5 shows a further embodiment with a tubular interior space in a cross-sectional view vertical to the longitudinal axis of the tube;

FIG. 6 shows an embodiment of the invention with a first and a second membrane, which are connected according to a blister packaging-type.

### DESCRIPTION ON THE BASIS OF EMBODIMENTS

The functional principle of a first embodiment of the invention is explained in FIG. 1. A vessel 1 comprises a first chamber 2 for nutrient medium and a second chamber 3, in which the cellulose-forming organisms, e.g. bacteria of the strain Gluconacetobacter xylinus, are present. The two chambers are separated by a membrane 4 made of hydrophilic PES. The first chamber 2 is completely filled with nutrient medium, wherein new nutrient medium is continuously flowing in via the supply connection 5 and nutrient medium is flowing off via the discharge connection 6. In this manner, the reaction conditions concerning the nutrient medium, i.e. in particular the composition and the pH of the culture medium can be controlled. At those points, where the membrane 4 is permeable for the nutrient medium, the nutrient medium penetrates, preferably at least co-driven by the capillary effect, the membrane 4 and thus gets into the second chamber 3, where it passes through the currently forming cellulose body 7 by means of diffusion to its surface and there supplies the cellulose-forming bacteria.

The nutrient solution e.g. consists of 20 g of glucose, 5 g of yeast extract, 5 g of bacto-peptone, 2.7 g of sodium phosphate and 1.15 g of citric acid monohydrate, 0.5 g of magnesium sulfate heptahydrate in one liter of water and has a pH of about 6.0. Furthermore, an oxygen-containing gas mixture to supply the cellulose-forming organisms flows through the second chamber 3 via the gas supply connection 8 and the gas discharge connection 9.

An apparatus working according to the principle described is the vessel 1 shown in FIG. 2. The first chamber 2 and the second chamber 3 comprise identical, semi-closed cylinders made of glass provided with flanges 10, 11, wherein the flanges 10, 11 are provided with polished surfaces 12, 13. Between the polished surfaces 12, 13, the membrane (not shown) is clamped with a flat silicon gasket (not shown).

Via the supply and discharge connections 5, 6, respectively arranged at an angle of 180° to one another, during operation, nutrient medium continuously flows through the first chamber 2 such that the circular part of the membrane 4 facing the interior of the first chamber 2 is completely wetted by the nutrient medium. During operation, sterile air flows through the second chamber 3 via the supply 8 and discharge connections 9, the partial water vapor pressure of which is adjusted to a value of 0.2 bars.

Prior to operation, the vessel 1 is steam-sterilized with the membrane clamped in, and then the side of the membrane 4 facing the second chamber is flushed with a suspension of Gluconacetobacter xylinus from a pre-culture, e.g. a 3-day old pre-culture of Gluconacetobacter xylinus (DSMZ Braunschweig), under clean and germ-free conditions, preferably at a clean-bench, such that a substantially uniform coverage of the membrane surface with the bacteria is achieved. In that, the medium flows via the hydrophilic membrane 4 into the first chamber 2 of the vessel 1, the bacteria, however, are retained by the membrane 4 in the second chamber 3. Then the vessel 1 is closed under the clean-bench, placed into an incubator, in order to control the reaction temperature, and then connected with sterile couplings at the supply and discharge connections with supply lines for air and the nutrient medium. In that, the first chamber 2 is operated in bypass to a stirrer tank reactor, using which the reaction conditions in the nutrient medium, in particular the pH, can be easily controlled. The formation of the cellulose body 7 in the second chamber 3 is visually observed via the transparent glass cylinder. Following 10 days, the vessel 1 is opened, the formed cellulose body 7 is detached from the surface of the membrane 4 and subsequently cleaned in boiling water and thereafter sterilized.

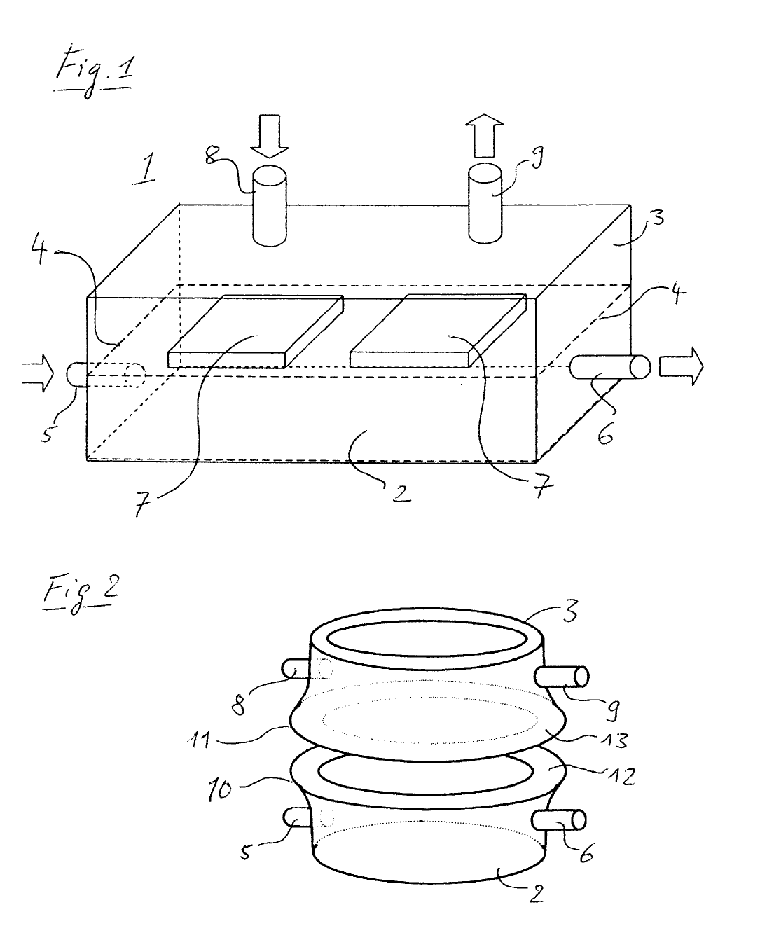

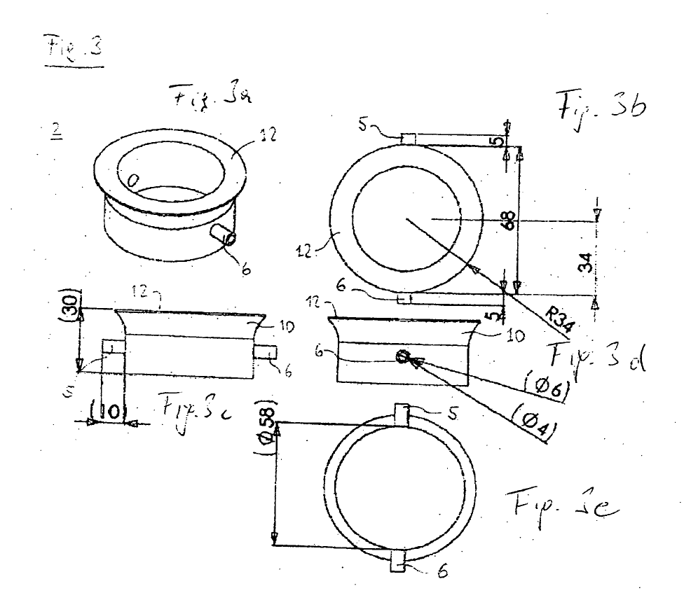

A further embodiment of the invention is shown in FIG. 4. In that, the membrane 4 is closed into an elongated circular cylinder, which in FIG. 4 is shown in cross-section. The circular cylinder is completely immersed into a reservoir of nutrient medium. The interior of the circular cylinder is inoculated with the cellulose-forming organisms, and through the membrane 4, the nutrient medium diffuses into the interior of the cylinder, such that a likewise circular cylinder-shaped hollow cellulose body 7 is formed on the inside of the cylinder. In order to supply the organisms with oxygen, air flows through the circular cylinder. In order to prevent the circular cylinder from filling up with nutrient medium, excess pressure prevails in the circular cylinder. Furthermore, the circular cylinder is slowly rotated in the direction of the arrow 14 in order to balance hydrostatic pressure differences and the unequal influence of gravity on the cellulose growth.

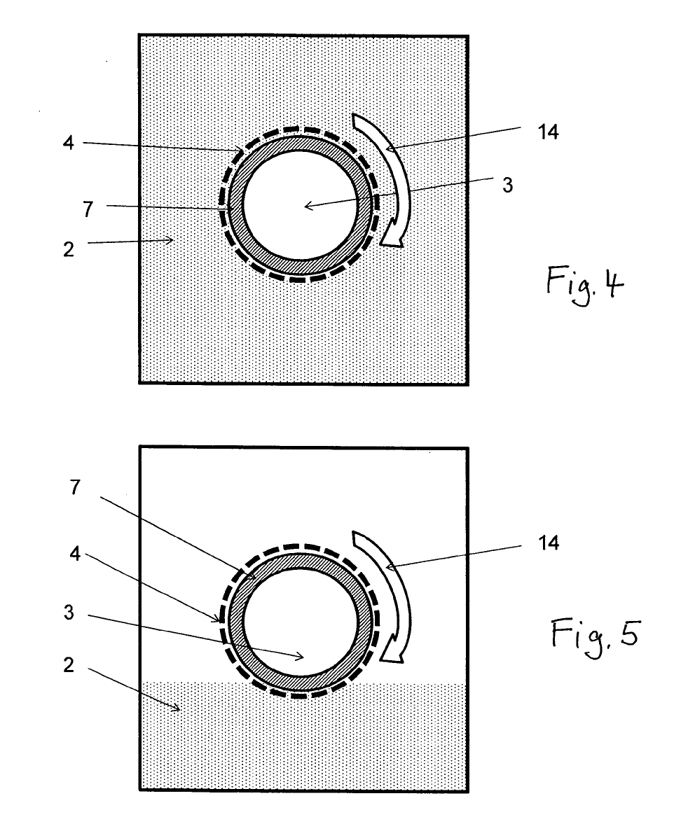

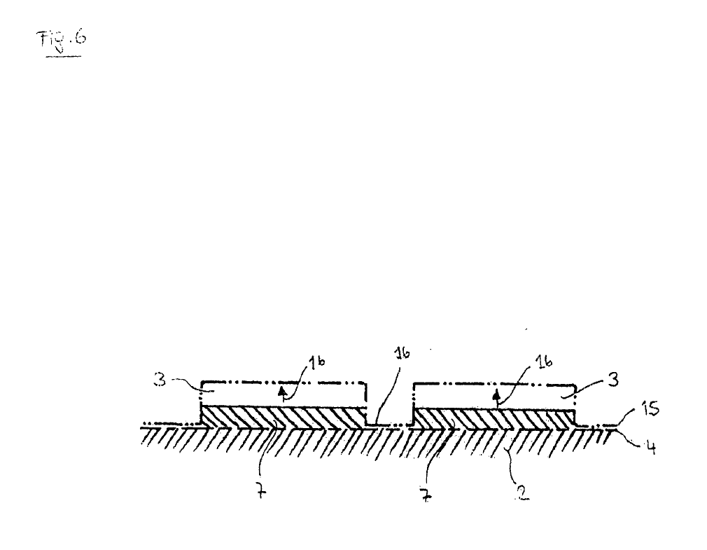

One variant of the embodiment described last is shown in FIG. 5. Here, the cylinder is not completely immersed into the reservoir 2 of the culture medium, but only partially. Therewith, it is no longer required to provide for excess pressure in the interior of the cylinder. Here, too, the cylinder is slowly rotated, such that all areas of the membrane 4 get in contact with the nutrient medium and hydrostatic pressure differences as well as the unequal influence of gravity on the cellulose growth can be balanced.

A further embodiment of the invention is shown in FIG. 6. Here, several second chambers 3 are provided, which are respectively formed by the membrane 4 and a second membrane 15. For this purpose, the second membrane 15, in other possible variants also the first membrane 4, or both membranes, are provided with several bulges forming the second chambers 3, and the first membrane 4 and the second membrane 15 are glued together in the areas 16 between these bulges. The second chambers 3 form hollow molds, into which the cellulose 7 grows, until the second chambers 3 are completely filled with the cellulose 7. Nutrient medium penetrates from a reservoir 2 of nutrient medium through the membrane 4 into the second chamber 3, where it diffuses through the cellulose body 7 to its surface, where the cellulose-forming organisms are located. The membrane 15 is gas-permeable, so that the organisms can be supplied with the required oxygen from the outside through the membrane 15. The direction of growth of the cellulose bodies is designated as 16.

The characteristics disclosed in the above description, the claims and the drawings may be of significance for the realization of the invention in its various embodiments individually as well as in any combination.

1. A method for producing a body containing cellulose (7) with the help of cellulose-producing organisms, which are supplied with a nutrient solution, the method comprising:
providing a membrane (4), which is permeable for said nutrient solution and substantially impermeable for said organisms,
providing said nutrient solution on a first side of said membrane (4), so that it can penetrate and pass through said membrane (4) to a second side thereof,
providing a gas atmosphere on the second side of said membrane (4), and
providing said cellulose-producing organisms on the second side of said membrane (4), so that said organisms are supplied with said nutrient solution passing through said membrane (4).

2. The method according to claim 1, wherein said membrane (4) is a ceramic membrane.

3. The method according to claim 1, wherein said membrane (4) is arranged as a flat layer, wherein said nutrient solution is substantially arranged below said membrane (4) and said cellulose-producing organisms above said membrane (4).

4. The method according to claim 1, wherein said membrane (4) forms a closed circumference of a hollow mold.

5. The method according to claim 4, wherein said cellulose-forming organisms are arranged in the interior of the hollow mold.

6. The method according to claim 1, wherein during the production process there is an exchange of said nutrient solution.

7. The method according to claim 1, wherein during the production process there is an exchange of the gas atmosphere.

8. The method according to claim 1, wherein the passage of nutrient solution through said membrane (4) is controlled by means of the gas pressure or a partial gas pressure.

9. The method according to claim 1, wherein the passage of nutrient solution through said membrane (4) is controlled by means of the hydrostatic pressure of the nutrient solution at said membrane (4).

10. The method according to claim 1, wherein on a side of said cellulose-producing organisms a second membrane (15) is provided, which is substantially impermeable for the organisms and the cellulose, and is permeable for a gas or a component of the gas.

11. The method according to claim 10, wherein said second membrane (15) together with the first membrane (4) defines a hollow space, in which said cellulose body (7) grows.

12. A body made of cellulose, produced according to the method according to any of claim 1.

13. An arrangement for producing a body containing cellulose with the help of cellulose-forming organisms, which are supplied with a nutrient solution, the arrangement comprising:
a vessel (1) with a membrane (4), which is substantially impermeable for said organisms and said cellulose, and is permeable for said nutrient solution.

14. The arrangement according to claim 13,
wherein said vessel (1) has at least one opening (5,6;8,9) for an exchange of gas and/or nutrient solution.

15. The arrangement according to claim 14, wherein said vessel (1) is closed, apart from said at least one optionally existing opening (5,6;8,9) for an exchange of gas and/or nutrient solution.

16. The arrangement according to claim 13, further comprising a second membrane (15), which in connection with said first membrane (4) defines one or several hollow spaces, in which the cellulose body or bodies (7), respectively, grow(s).
